(19) United States
(12) Patent Application Publication
(10) Pub. No.: US 2010/0158897 A1
(43) Pub. Date: Jun. 24, 2010

(54) PAI-1 MODULATORS FOR THE TREATMENT OF OCULAR DISORDERS

(75) Inventors: Debra L. Fleenor, Crowley, TX (US); Allan R. Shepard, Fort Worth, TX (US)

Correspondence Address: ALCON, IP LEGAL, TB4-8, 6201 SOUTH FREEWAY, FORT WORTH, TX, 76134, US

(73) Assignee: ALCON RESEARCH, LTD., Fort Worth, TX (US)

(21) Appl. No.: 12/716,137

(22) Filed: Mar. 2, 2010

Related U.S. Application Data
(63) Continuation-in-part of application No. 11/931,393, filed on Oct. 31, 2007.
Application No. 11/931,393 is a continuation-in-part of application No. 12/421,456, filed on Apr. 9, 2009.
Application No. 12/421,456 is a continuation-in-part of application No. 11/931,393, filed on Oct. 31, 2007.
(60) Provisional application No. 60/863,715, filed on Oct. 31, 2006.
(60) Provisional application No. 61/048,176, filed on Apr. 26, 2008.
(60) Provisional application No. 60/863,715, filed on Oct. 31, 2006.

(51) Int. Cl. A61K 31/4709; A61K 39/395; A61P 27/02; A61P 27/06

(52) U.S. Cl. 424/130.1; 514/312

(57) ABSTRACT

The invention concerns in one embodiment a method for treating glaucoma or elevated IOP in a patient comprising administering to the patient an effective amount of a composition comprising an agent that modulates PAI-1 activity. In a preferred embodiment, the agent that modulates PAI-1 expression and/or activity is cilostazol or an analog or metabolite of cilostazol.

### CROSS-REFERENCE TO RELATED APPLICATION

This application is a continuation in part of U.S. patent application Ser. No. 11/931,393 filed Oct. 31, 2007 which claims priority to U.S. Provisional Patent Application No. 60/863,715 filed Oct. 31, 2006 and is also a continuation-in-part of U.S. patent application Ser. No. 12/421,456 filed Apr. 9, 2009 which claims priority to 61/048,176 filed Apr. 26, 2008 and is a continuation in part of U.S. patent application Ser. No. 11/931,393 filed Oct. 31, 2007 which claims priority to U.S. Provisional Patent Application No. 60/863,715 filed Oct. 31, 2006, the entire contents of which are incorporated herein by reference.

### TECHNICAL FIELD OF THE INVENTION

The present invention is generally related to treatments for ocular disorders and more specifically to the use of agents that lower IOP and/or treat or prevent glaucoma.

### BACKGROUND OF THE INVENTION

Primary open angle glaucoma (POAG), also known as chronic or simple glaucoma, represents the majority of all glaucomas in the United States. Most forms of glaucoma result from disturbances in the flow of aqueous humor that have an anatomical, biochemical or physiological basis.

Elevated levels of plasminogen activator inhibitor-1 (PAI-1) have been detected in the aqueous humor of glaucoma patients (Dan et al., Arch Opthalmol, Vol. 123:220-224, 2005). PAI-1 levels are increased by the cytokine TGFβ (Binder et al., News Physiol Sci, Vol. 17:56-61, 2002), among other endogenous stimuli. PAI-1 inhibits the activity of both tissue plasminogen activator (tPA) and urokinase plasminogen activator (uPA). Both tPA and uPA catalyze the conversion of plasminogen into plasmin, a key intermediate in the fibrinolytic cascade (Wu et al., Curr Drug Targets, Vol. 2:27-42, 2002). Plasmin is known to promote the conversion of certain pro-matrix metalloproteinases (MMPs) into their active, extracellular matrix (ECM)-degrading, forms (He et al., PNAS, Vol. 86:2632-2636, 1989). PAI-1 also modulates the association of vitronectin, an ECM component, with cell surface integrins which act as adhesion receptors (Zhou et al., Nature Structural Biology, Vol. 10(7):541-544, 2003). Thus, PAI-1 has been linked to both decreased adhesion and increased detachment of cells in non-ocular tissues.

Drug therapies that have proven to be effective for the reduction of IOP (IOP) and/or the treatment of POAG include both agents that decrease aqueous humor production and agents that increase the outflow facility. Such therapies are in general administered by one of two possible routes; topically (direct application to the eye) or orally. However, pharmaceutical ocular anti-hypertension approaches have exhibited various undesirable side effects. For example, miotics such as pilocarpine can cause blurring of vision, headaches, and other negative visual side effects. Systemically administered carbonic anhydrase inhibitors can also cause nausea, dyspepsia, fatigue, and metabolic acidosis. Certain prostaglandins cause hyperemia, ocular itching, and darkening of eyelashes and periorbital skin. Such negative side-effects may lead to decreased patient compliance or to termination of therapy such that vision continues to deteriorate. Additionally, there are individuals who simply do not respond well when treated with certain existing glaucoma therapies. There is, therefore, a need for other therapeutic agents for the treatment of ocular disorders such as glaucoma and ocular hypertension.

Cilostazol (Pletal®) has been approved in the United States since 1999 for the treatment of intermittent claudication, i.e., leg pain due to compromised blood flow associated with peripheral vascular disorders. It has also shown efficacy in clinical trials as an inhibitor of restenosis after coronary stent placement. In the eye, cilostazol has been shown to increase the survival of retinal ganglion cells post-axotomy (Kashimoto et al., Neuroscience Letters, Vol. 436:116-119, 2008), and has been reported to enhance blood flow to the optic nerve head and retina (Suzuki et al., J. Ocular Therapy, Vol. 14(3):239-245, 1998), but has not been contemplated as a therapeutic for glaucoma or ocular hypertension. Cilostazol reportedly suppresses transforming growth factor (TGFβ2) and plasminogen activator inhibitor-1 (PAI-1) protein expression, as well as PAI-1 activity, in non-ocular tissues of rats (Mohamed, Biomed Pharmacother. 2009 Mar. 12. [Epub ahead of print]; Lee et al, Atherosclerosis, Vol. 207:391-398, 2009).

### BRIEF SUMMARY OF THE INVENTION

Embodiments of the present invention recognize that the modulation of PAI-1 can be used to treat ocular disease and/or lower IOP. One embodiment provides a method for treating glaucoma or elevated IOP in a patient comprising administering to the patient an effective amount of a composition comprising an agent that modulates PAI-1.

Another embodiment of the present invention is a method of treating a PAI-1-associated ocular disorder comprising administering an effective amount of a composition comprising an agent that modulates PAI-1 binding to vitronectin.

A preferred embodiment of the present invention is a method for treating glaucoma or elevated IOP in a patient comprising administering to the patient an effective amount of a composition comprising cilostazol or an analog or metabolite thereof such as 3,4-dehydro cilostazol or cilostamide.

In certain of these embodiments, the agent is ZK4044, PAI-039, WAY-140312, HP-129, T-686, XR5967, XR334, XR330, XR5118, PAI-1 antibodies, PAI-1 peptidomimetics, and combinations thereof.

Yet another embodiment is a method of manufacturing a compound to be used as a treatment for glaucoma or elevated IOP comprising providing a candidate substance suspected of modulating PAI-1, selecting the compound by assessing the ability of the candidate substance to decrease the amount of total and/or active PAI-1 in the trabecular meshwork of a subject suffering from glaucoma or elevated PAI-1, and manufacturing the selected compound.

In certain embodiments, compositions of the invention further comprise a compound selected from the group consisting of opthalmologically acceptable preservatives, surfactants, viscosity enhancers, penetration enhancers, gelling agents, hydrophobic bases, vehicles, buffers, sodium chloride, water, and combinations thereof.

In yet other embodiments, a compound selected from the group consisting of β-blockers, prostaglandin analogs, carbonic anhydrase inhibitors, α₂ agonists, miotics, neuroprotectants, rho kinase inhibitors, and combinations thereof may be administered either as part of the composition or as a separate administration.

The foregoing brief summary broadly describes the features and technical advantages of certain embodiments of the present invention. Additional features and technical advantages will be described in the detailed description of the invention that follows. Novel features which are believed to be characteristic of the invention will be better understood from the detailed description of the invention when considered in connection with any accompanying figures. However, figures provided herein are intended to help illustrate the invention or assist with developing an understanding of the invention, and are not intended to be definitions of the invention's scope.

### BRIEF DESCRIPTION OF THE DRAWINGS

A more complete understanding of the present invention and the advantages thereof may be acquired by referring to the following description, taken in conjunction with the accompanying drawing and wherein:

FIG. 1 is a graph of experimental results showing the concentration-dependent effect of TGFβ2 (24 h) on levels of PAI-1 in human trabecular meshwork (GTM-3) cell supernatants. Data are expressed as mean and SEM, n=3. *p<0.05 versus corresponding vehicle group by one-way ANOVA, followed by the Dunnett test;

FIG. 2 is a graph of experimental results showing PAI-1 levels in GTM-3 cell supernatants with or without treatment with TGFβ2 (5 ng/mL) for various time periods. Data are expressed as mean and SEM, n=3. *p<0.05 versus corresponding vehicle time point group, by Student's t-test;

FIG. 3 is a bar graph showing the effect of wild-type PAI-1 (1 μg/mL, 2 h) and TGFβ2 (5 ng/mL, 2 h) on adhesion of transformed (GTM-3) and non-transformed (GTM730) cells to vitronectin substrate. Data are expressed as mean and SEM, n=12-44. *p<0.05 versus corresponding untreated groups by one-way ANOVA, followed by the Dunnett test;

FIG. 4 is a graph of experimental results showing concentration-dependent effect of wild-type PAI-1 (2 h) on adhesion of GTM-3 cells to vitronectin substrate. Data are expressed as mean and SEM, n=4. *p<0.05 versus vehicle group by one-way ANOVA, followed by the Dunnett test;

FIG. 5 is a graph of experimental results showing the time-dependent effect of wild-type PAI-1 (1 μg/mL) on adhesion of GTM-3 cells to vitronectin substrate. Data are expressed as mean and SEM, n=12-44;

FIG. 6 is a bar graph of experimental results showing the effect of wild-type PAI-1 (1 μg/mL, 1 h) versus a stable, degradation-resistant PAI-1 mutant (1 μg/mL, 1 h) on adhesion of GTM-3 and GTM730 cells to vitronectin substrate. Data are expressed as mean and SEM, n=4. *p<0.05 versus corresponding untreated groups by Student's t-test. **p<0.05 versus corresponding PAI-1 (wild-type) treated groups by Student's t-test;

FIG. 7 is a bar graph of experimental results showing the effect of wild-type PAI-1 (1 μg/mL, 2 h) versus a non-vitronectin binding PAI-1 mutant (1 μg/mL, 2 h) on adhesion of GTM-3 cells to vitronectin substrate. Data are expressed as mean and SEM, n=4-24. *p<0.05 versus untreated group by one-way ANOVA, followed by the Dunnett test;

FIG. 8 is a graph of experimental results showing the concentration-dependent effect of wild-type PAI-1 (4 h) on migration of GTM-3 cells. Data are expressed as mean and SEM, n=4-32. *p<0.05 versus vehicle group by one-way ANOVA, followed by the Dunnett test;

FIGS. 9a-9c are graphs showing the effect of cilostazol on TGFβ2-induced (5 ng/mL; 24 h) total PAI-1 protein in supernatants from three different human trabecular meshwork (HTM) cell lines;

FIG. 10 is a graph showing the effect of the cilostazol metabolite 3,4-dehydro cilostazol (“DHC”) on TGFβ2-induced (5 ng/mL; 24 h) total PAI-1 protein in supernatants from GTM-3 HTM cells;

FIG. 11 is a graph showing the effect of the cilostazol analog cilostamide on TGFβ2-induced (5 ng/mL; 24 h) total PAI-1 protein in supernatants from GTM-3 HTM cells; and

FIGS. 12a-12b are graphs showing the effect of topical ocular administration of various concentrations of cilostazol compared to control on Ad.TGFβ2-induced ocular hypertension in mice.

### DETAILED DESCRIPTION OF THE INVENTION

PAI-1 has been linked to both decreased adhesion and increased detachment of cells in non-ocular tissues. A review of the data disclosed herein leads to the conclusion that increased PAI-1 levels in glaucomatous aqueous humor may be due to actions of TGFβ2 on trabecular meshwork cells. PAI-1-induced decreases in TM cell adhesion are likely due to PAI-1 interference with attachment of cells to the extracellular matrix component vitronectin. Additionally, the PAI-1-induced decrease in TM cell adhesion may facilitate migration of TM cells from the meshwork environment. Thus, the PAI-1 induced decrease in TM cell adhesion and increase in TM cell migration may be important factors in the decrease of TM cellularity seen in glaucomatous eyes. Certain embodiments of the present invention recognize that PAI-1 may cause such effects in trabecular meshwork (TM) tissues.

Circulating PAI-1 normally exists in a latent form, due to the ability of the active PAI-1 to rapidly and spontaneously transform to its inactive conformation. However, PAI-1 bound to vitronectin becomes stabilized in its active form, resulting in a much longer half-life. Thus, one means to reduce deleterious effects of active PAI-1 is to utilize agents which modulate the interaction of PAI-1 and vitronectin. Such agents would thus allow unbound vitronectin in the ECM to associate with its cell surface (integrin) receptors, thus enhancing cellular adhesion and reducing cell loss from TM tissues. Modulation of PAI-1's expression/activity and or its ability to bind vitronectin can provide a viable therapeutic approach to the management of glaucoma.

Certain embodiments of the present invention are methods for targeting the downstream effects of PAI-1 in ocular disorders such as glaucoma by interfering with the binding of PAI-1 to vitronectin as shown in the following scheme,

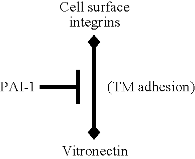

where PAI-1 decreases binding of trabecular meshwork (TM) cell surface adhesion receptors (integrins) to vitronectin, an extracellular matrix component. As a consequence, cells detach from the TM and are swept via aqueous flow into the juxtacanulicular region of TM. This accumulation of detached TM cells and their debris contributes to increased outflow resistance and elevated IOP. Modulation of PAI-1 binding to vitronectin can thus decrease the detachment of TM cells and reduce increased outflow resistance and elevated IOP. Additionally, TM tissue cellularity may be thereby increased, preserving such vital functions as phagocytosis.

### PAI-1 Modulators

Various PAI-1 binding modulators are known in the art. Jensen et al, for example, describe the discovery of a small peptide with strong affinity for wild-type PAI-1 and which inhibits association of the uPA-PAI-1 complex with low density lipoprotein receptor family members (Jensen et al, Inhibition of plasminogen activator inhibitor-1 binding to endocytosis receptors of the low-density-lipoprotein receptor family by a peptide isolated from a phage display library, Biochem J., Vol. 399(3):387-396, 2006). Agents that alter PAI-1's ability to inhibit tissue plasminogen activator (tPA) and/or urokinase plasminogen activator (uPA) may modulate PAI-1 binding as well. Such agents include, but are not limited to, ZK4044 (Liang et al., Characterization of a small molecule PAI-1 inhibitor, ZK4044, Thrombosis Research, Vol. 115(4):341-50, 2005), PAI-039 (tiplaxtinin) (Weisberg et al., Pharmacological inhibition and genetic deficiency of plasminogen activator inhibitor-1 attenuates angiotensin II/salt-induced aortic remodeling. Arterioscler Thrombosis Vasc Biology, Vol. 25(2):365-71, 2005 February; Hennan et al., Evaluation of PAI-039 [{1-benzyl-5-[4-(trifluoromethoxy)phenyl]-1H-indol-3-yl}(oxo)acetic acid], a novel plasminogen activator inhibitor-1 inhibitor, in a canine model of coronary artery thrombosis., J Pharmacol Exp Ther., Vol. 314(2):710-6, 2005 Aug. Epub, 2005 Apr. 28; Elokdah et al., A novel, orally efficacious inhibitor of plasminogen activator inhibitor-1: design, synthesis, and preclinical characterization. Journal Med. Chem., Vol. 47(14):3491-3494, 2004 Jul. 1), WAY140312 (Crandall et al., Characterization and comparative evaluation of a structurally unique PAI-1 inhibitor exhibiting oral in-vivo efficacy., J Thromb Haemost., Vol. 2(8):1422-1428, August 2004; Crandall et al., WAY-140312 reduces plasma PAI-1 while maintaining normal platelet aggregation, Biochem Biophys Res Commun., Vol. 311(4):904-8, 2003 Nov. 28), HP129 (fendosal) (Ye et al., Synthesis and biological evaluation of menthol-based derivatives as inhibitors of plasminogen activator inhibitor-1 (PAI-1). Bioorg Med Chem. Lett., Vol. 13(19):3361-3365, 2003 Oct. 6), and T-686 (Murakami et al., Protective effect of T-686, an inhibitor of plasminogen activator inhibitor-1 production, against the lethal effect of lipopolysaccharide in mice, Japan Journal Pharmacol., Vol. 75(3):291-294. 1997 November); Ohtani et al., T-686, a novel inhibitor of plasminogen activator inhibitor-1, inhibits thrombosis without impairment of hemostasis in rats. Eur J. Pharmacol., Vol. 330(2-3):151-156, 1997 Jul. 9; Vinogradsky et al., A new butadiene derivative, T-686, inhibits plasminogen activator inhibitor type-1 production in vitro by cultured human vascular endothelial cells and development of atherosclerotic lesions in vivo in rabbits, Thrombosis Research., Vol. 85(4):305-14, 1997 Feb. 15; Ohtani et al., Inhibitory effect of a new butadiene derivative on the production of plasminogen activator inhibitor-1 in cultured bovine endothelial cells, Journal Biochem (Tokyo), Vol. 120(6):1203-8, 1996 Dec.). Bryans et al., Inhibition of plasminogen activator inhibitor-1 activity by two diketopiperazines, XR330 and XR334, The Journal of Antibiotics, Vol. 49(10):1014-1021, 1996 October. Einholm et al., Biochemical mechanism of action of a diketopiperazine inactivator of plasminogen activator inhibitor-1, XR5118, Biochem Journal, Vol. 373:723-732, 2003.

Additionally, PAI-1 inhibitors such as those taught by Ye (Ye et al., Synthesis and biological evaluation of piperazine-based derivatives as inhibitors of plasminogen activator inhibitor-1 (PAI-1). Bioorg Med Chem. Lett., Vol. 14(3):761-5, 2004 Feb. 9; Ye et al., Synthesis and biological evaluation of menthol-based derivatives as inhibitors of plasminogen activator inhibitor-1 (PAI-1). Bioorg Med Chem. Lett., Vol. 13(19):3361-3365, 2003 Oct. 6) and antibody-based inhibitors such as those taught by Verbeke (Verbeke et al., Cloning and paratope analysis of an antibody fragment, a rational approach for the design of a PAI-1 inhibitor. Journal Thromb Haemost., Vol. 2(2):289-297, 2004 February) and van Giezen (van Giezen et al., The Fab-fragment of a PAI-1 inhibiting antibody reduces thrombus size and restores blood flow in a rat model of arterial thrombosis. Thromb Haemost., Vol. 77(5):964-969, May 1997) may also modulate PAI-1 binding. Other PAI-1 modulators may comprise PAI-1 peptidomimetics.

As discussed above, cilostazol has been shown to inhibit PAI-1 expression and activity in rat tissue (Mohamed, Biomed Pharmacother., 2009 Mar. 12. [Epub ahead of print]; Lee et al, Atherosclerosis, Vol. 207:391-398, 2009), and is a preferred PAI-1 modulator of the present invention. The structure of cilostazol is shown below:

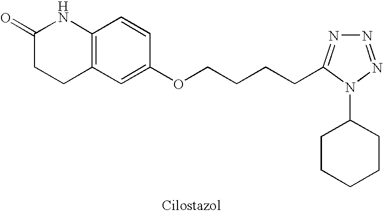

Cilostazol (OPC-13013; CAS No. 73963-72-1) is metabolized by liver enzymes (primarily CYP3A4) to active metabolites such as 3,4-dehydro-cilostazol (DHC) and 4′-trans-hydroxy-cilostazol (HC). HC is much less active than cilostazol. However, the activity of DHC(OPC-13015; CAS No. 73963-62-9) is reportedly 4-7 fold greater than cilostazol, and may account for ≧50% of total activity attributed to cilostazol. The structure of 3,4-dehydro-cilostazol (DHC) is shown below:

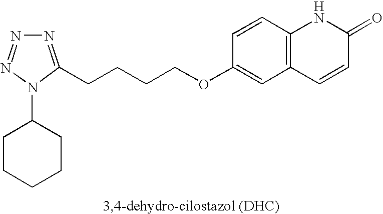

Cilostamide (OPC-3689; CAS No. 6855-75-4), is a cilostazol analog. The structure of cilostamide is shown below:

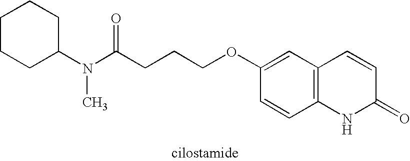

The contents of all references cited in this section under heading “PAI-1 Modulators” are hereby incorporated by reference in their entirety.

### Modes of Delivery

The PAI-1 modulators of the present invention can be incorporated into various types of ophthalmic formulations for delivery. The compounds may be delivered directly to the eye (for example: topical ocular drops or ointments; slow release devices such as pharmaceutical drug delivery sponges implanted in the cul-de-sac or implanted adjacent to the sclera or within the eye; periocular, conjunctival, sub-tenons, intracameral, intravitreal, or intracanalicular injections) or systemically (for example: orally, intravenous, subcutaneous or intramuscular injections; parenteral, dermal or nasal delivery) using techniques well known by those of ordinary skill in the art. It is further contemplated that the PAI-1 modulators of the invention may be formulated in intraocular inserts or implantable devices.

The PAI-1 modulators disclosed herein are preferably incorporated into topical ophthalmic formulations for delivery to the eye. The compounds may be combined with opthalmologically acceptable preservatives, surfactants, viscosity enhancers, penetration enhancers, buffers, sodium chloride, and water to form an aqueous, sterile ophthalmic suspension or solution. Ophthalmic solution formulations may be prepared by dissolving a compound in a physiologically acceptable isotonic aqueous buffer. Further, the ophthalmic solution may include an opthalmologically acceptable surfactant to assist in dissolving the compound. Furthermore, the ophthalmic solution may contain an agent to increase viscosity such as hydroxymethylcellulose, hydroxyethylcellulose, hydroxypropylmethylcellulose, methylcellulose, polyvinylpyrrolidone, or the like, to improve the retention of the formulation in the conjunctival sac. Gelling agents can also be used, including, but not limited to, gellan and xanthan gum. In order to prepare sterile ophthalmic ointment formulations, the active ingredient is combined with a preservative in an appropriate vehicle such as mineral oil, liquid lanolin, or white petrolatum. Sterile ophthalmic gel formulations may be prepared by suspending the compound in a hydrophilic base prepared from the combination of, for example, carbopol-974, or the like, according to the published formulations for analogous ophthalmic preparations; preservatives and tonicity agents can be incorporated.

PAI-1 modulators are preferably formulated as topical ophthalmic suspensions or solutions, with a pH of about 4 to 8. The compounds are contained in the topical suspensions or solutions in amounts sufficient to lower IOP in patients experiencing elevated IOP and/or maintaining normal IOP levels in glaucoma patients. Such amounts are referred to herein as “an amount effective to control IOP,” or more simply “an effective amount.” The compounds will normally be contained in these formulations in an amount 0.01 to 5 percent by weight/volume (“w/v %”), but preferably in an amount of 0.25 to 2 w/v %. Thus, for topical presentation 1 to 2 drops of these formulations would be delivered to the surface of the eye 1 to 4 times per day, according to the discretion of a skilled clinician. The PAI-1 modulators may also be used in combination with other elevated IOP or glaucoma treatment agents, such as, but not limited to, rho kinase inhibitors, β-blockers, prostaglandin analogs, carbonic anhydrase inhibitors, α₂ agonists, miotics, serotonergic agonists and neuroprotectants.

As used herein, “PAI-1 modulator” encompasses such modulators as well as their pharmaceutically-acceptable salts. A pharmaceutically acceptable salt of a PAI-1 modulator is a salt that retains PAI-1 modulatory activity and is acceptable by the human body. Salts may be acid or base salts since agents herein may have amino or carboxy substituents. A salt may be formed with an acid such as acetic acid, benzoic acid, cinnamic acid, citric acid, ethanesulfonic acid, fumaric acid, glycolic acid, hydrobromic acid, hydrochloric acid, maleic acid, malonic acid, mandelic acid, methanesulfonic acid, nitric acid, oxalic acid, phosphoric acid, propionic acid, pyruvic acid, salicylic acid, succinic acid, sulfuric acid, tartaric acid, p-toluenesulfonic acid, trifluoroacetic acid, and the like. A salt may be formed with a base such as a primary, secondary, or tertiary amine, aluminum, ammonium, calcium, copper, iron, lithium, magnesium, manganese, potassium, sodium, zinc, and the like.

Determination of Biological Activity PAI-1 modulators can be selected using binding assays or functional assays that can also be used to determine their biological activity. Such assays can be developed by those of skill in the art using previously described methods. Other assays are or can be derived from data presented infra in the Examples. For example, the TM cell migration assay later described can be used where a putative PAI-1 modulator is added as a test agent.

### In Vivo Biological Activity Testing

The ability of certain PAI-1 modulators to safely lower IOP may be evaluated in certain embodiments by means of in vivo assays using New Zealand albino rabbits and/or Cynomolgus monkeys.

### Ocular Safety Evaluation in New Zealand Albino Rabbits

Both eyes of New Zealand albino rabbits are topically dosed with one 30 μL aliquot of a test compound in a vehicle. Animals are monitored continuously for 0.5 hr post-dose and then every 0.5 hours through 2 hours or until effects are no longer evident.

### Acute IOP Response in New Zealand Albino Rabbits

Intraocular pressure (IOP) is determined with a Mentor Classic 30 pneumatonometer after light corneal anesthesia with 0.1% proparacaine. Eyes are rinsed with one or two drops of saline after each measurement. After a baseline IOP measurement, test compound is instilled in one 30 μL aliquot to one or both eye of each animal or compound to one eye and vehicle to the contralateral eye. Subsequent IOP measurements are taken at 0.5, 1, 2, 3, 4, and 5 hours.

### Acute IOP Response in Cynomolgus Monkeys

Intraocular pressure (IOP) is determined with an Alcon pneumatonometer after light corneal anesthesia with 0.1% proparacaine as previously described (Sharif et al., J. Ocular Pharmacol. Ther., Vol. 17:305-317, 2001; May et al., J. Pharmacol. Exp. Ther., Vol. 306:301-309, 2003). Eyes are rinsed with one or two drops of saline after each measurement. After a baseline IOP measurement, test compound is instilled in one or two 30 μL aliquots to the selected eyes of cynomolgus monkeys. Subsequent IOP measurements are taken at 1, 3, and 6 hours. Right eyes of all animals had undergone laser trabeculoplasty to induce ocular hypertension. All left eyes are normal and thus have normal IOP.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### TGFβ2 Increases PAI-1 Content in TM Cells

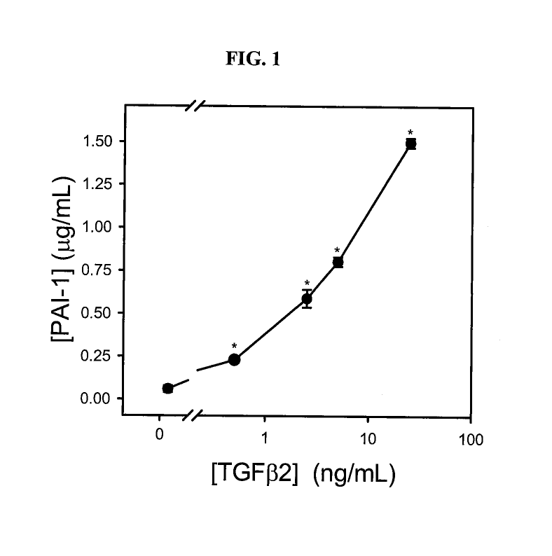

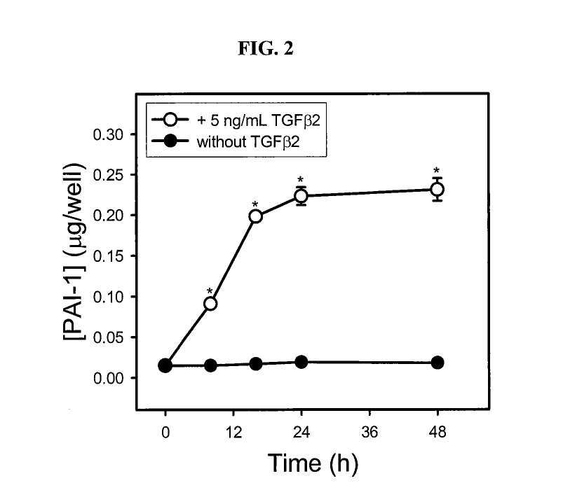

FIG. 1 presents the results of experiments showing that TGFβ2 increases the PAI-1 content in trabecular meshwork cell cultures (GTM-3). PAI-1 mediated effects may contribute to the previously observed TGFβ2-mediated accumulation of extracellular matrix materials in various tissues, including TM tissues. FIG. 2 demonstrates that such TGFβ2-mediated PAI-1 increases are persistent in cell cultures treated with TGFβ2. TGFβ2-treatment results in both concentration-dependent and time-dependent accumulation of PAI-1 in TM cell supernatants (FIGS. 1 and 2). PAI-1 levels increase gradually in response to TGFβ2, reaching a constant level at approximately 24 h post-treatment.

### Example 2

### Wild-Type PAI-1 Decreases Adhesion of TM Cells

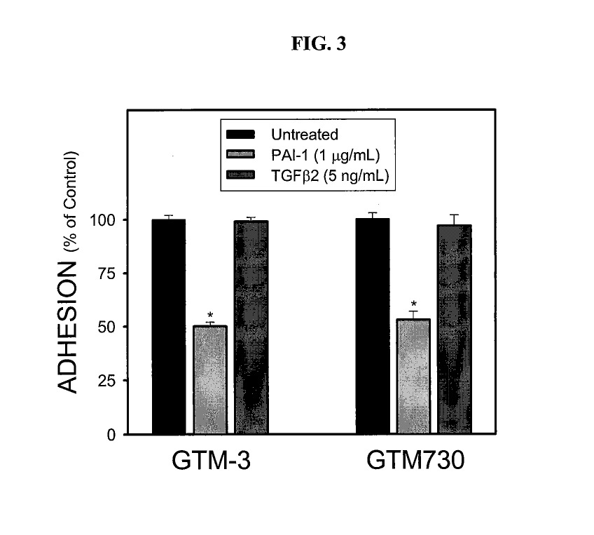

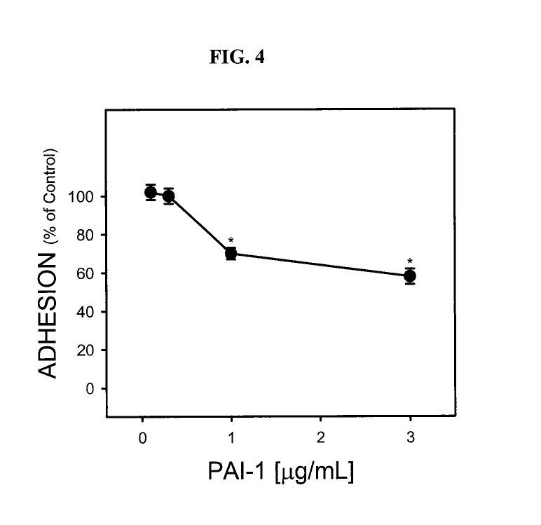

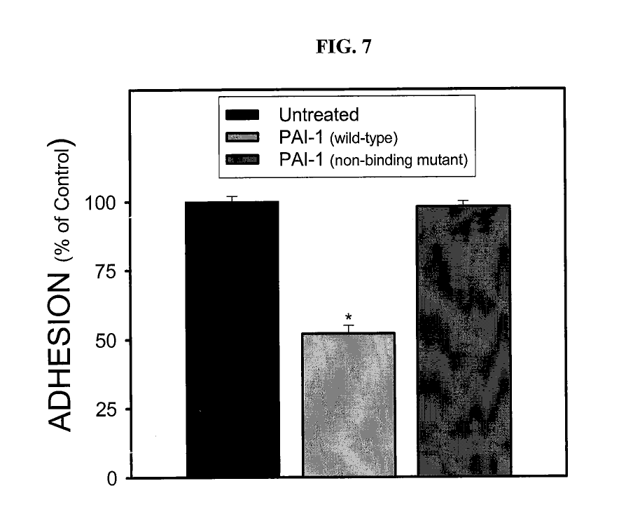

FIG. 3 presents experimental data demonstrating the ability of recombinant human PAI-1 (2 h treatment) to decrease adhesion of cultured human TM cells to a vitronectin substrate; in that same model, adhesion was not affected by a mutant PAI-1 which does not bind vitronectin (FIG. 7). FIG. 4 shows the effect of increasing concentrations of PAI-1 on TM cell adhesion. The effect of PAI-1 on adhesion was dose-dependent, with an estimated EC₅₀ of approximately 0.6 μM.

Such interference with TM cell adhesion may thereby trigger accelerated TM cell loss such as that seen in glaucoma, particularly POAG. Detached TM cells may contribute to the obstruction of aqueous humor outflow, a process believed to lead to increased outflow resistance and elevated IOP. Loss of TM cells from the meshwork tissues may also lead to impaired debris clearance, as a result of reduced phagocytic capacity.

Referring again to FIG. 3, cells that were treated with TGFβ2 for 2 hr did not experience measurable loss of adhesion when compared to controls. The lack of effect of short-term treatment with TGFβ2 is likely due to insufficient TGFβ2-mediated PAI-1 induction during the 2 hr treatment period (vis. FIG. 2). Responses of SV40-transformed (GTM-3) cells were highly similar to that of non-transformed (GTM730) cells.

### Example 3

### Wild-Type PAI-1 Degrades Over Time

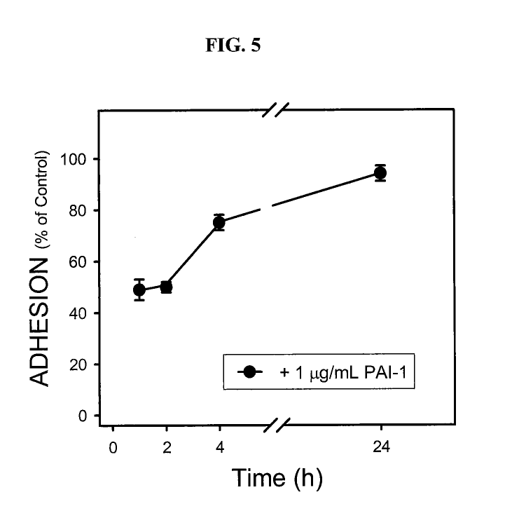

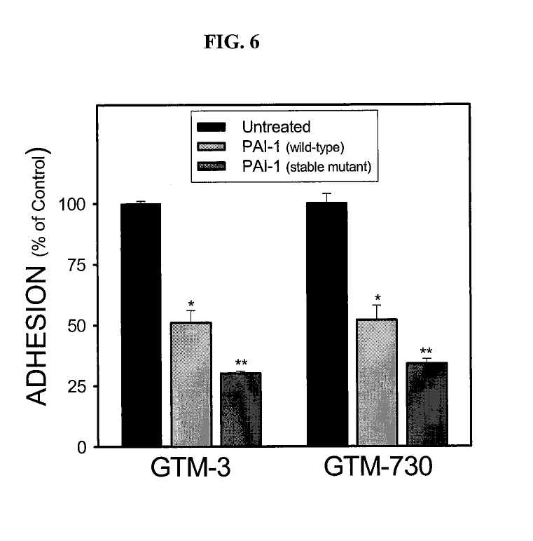

FIG. 5 shows experimental data indicating that the wild type PAI-1-mediated loss of adhesion is transient, with adhesion levels returning to near-control levels after 24 h. FIG. 6 is a bar graph of experimental results showing the effect of wild-type PAI-1 (1 μg/mL, 1 h) versus a stable, degradation-resistant PAI-1 mutant (1 μg/mL, 1 h) on adhesion of GTM-3 and GTM730 cells to vitronectin substrate. Taken in context with FIG. 5, the data demonstrate that wild-type PAI-1 appears to degrade over time. The effect of PAI-1 was therefore enhanced by use of a stable PAI-1 mutant (mixture of the K154T, Q139L, M354I, and H150H mutations) which is more degradation-resistant than the wild-type protein.

### Example 4

### Wild-Type PAI-1 Effects on Adhesion are Vitronectin-Mediated

FIG. 7 is a bar graph of experimental results showing the effect of wild-type PAI-1 (1 μg/mL, 2 h) versus a non-vitronectin binding PAI-1 mutant (1 μg/mL, 2 h) on adhesion of GTM-3 cells to vitronectin substrate. The mutant PAI-1, which does not bind vitronectin yet is known to be otherwise functional, was without effect on TM cell adhesion to vitronectin substrate, while the wild-type vitronectin-binding PAI-1 decreased adhesion to ca. 50% of control levels.

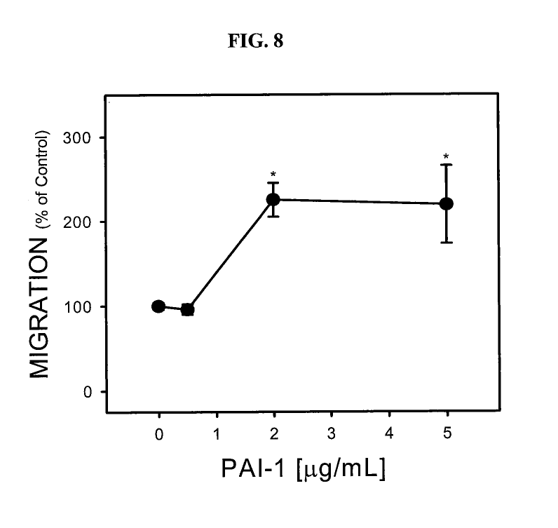

FIG. 8 is a graph of experimental results showing the concentration-dependent effect of wild-type PAI-1 (4 h) on migration of GTM-3 cells. Wild-type PAI-1, at concentrations similar to that which reduce TM cell adhesion, induced migration of TM cells.

### Example 5

### In Vitro and In Vivo Cilostazol, 3,4-Dehydro Cilostazol, and Cilostamide Experiments

Experiments were performed to examine the effects of cilostazol and a cilostazol analog and metabolite on PAI-1 expression. In addition, the intraocular pressure-lowering effects of cilostazol were examined in a mouse model.

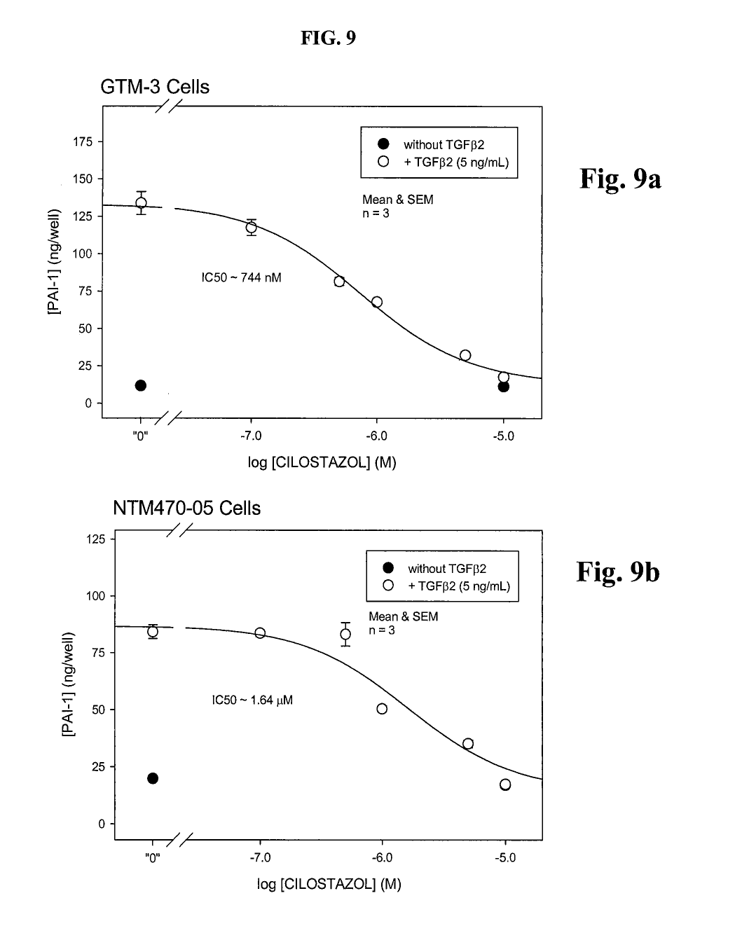

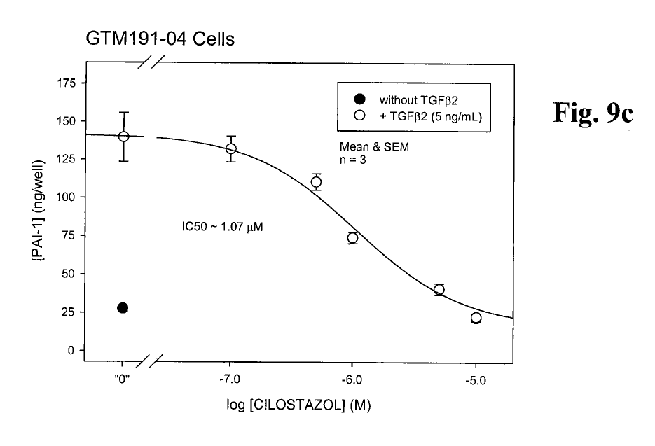

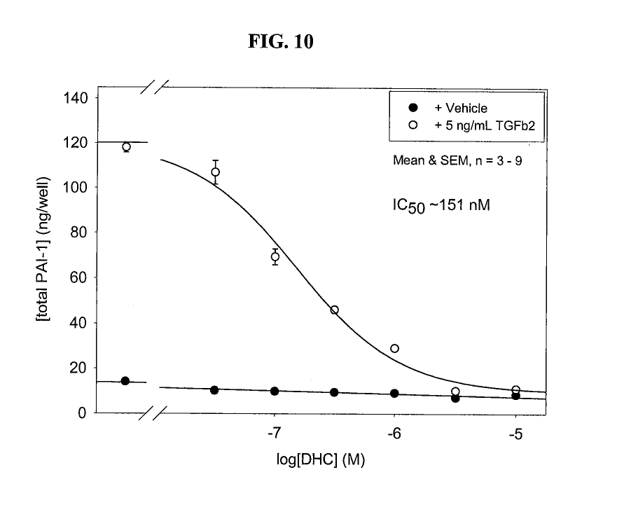

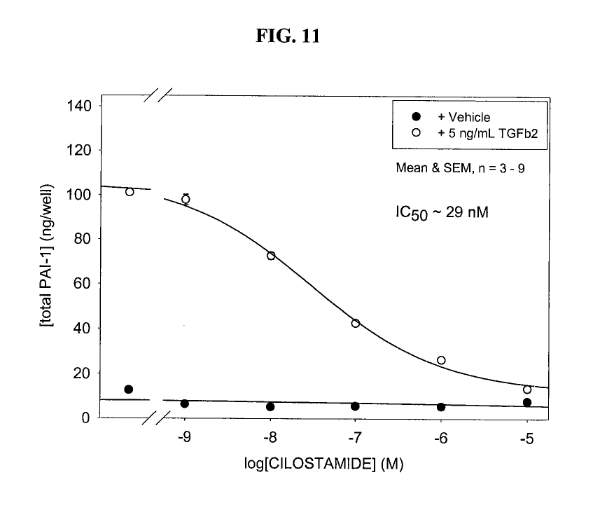

FIGS. 9-11 present graphs showing the effect of cilostazol, 3,4-dehydro cilostazol, and cilostamide on TGFβ2-induced (5 ng/mL; 24 h) total PAI-1 protein in supernatants from human trabecular meshwork (HTM) cell lines. Cilostazol was tested against three different HTM cell lines (GTM, NTM470-05, and GTM191-04). All graphs demonstrate a dose-dependent inhibitory effect on PAI-1 protein expression by the cell cultures for cilostazol and the cilostazol analog and metabolite.

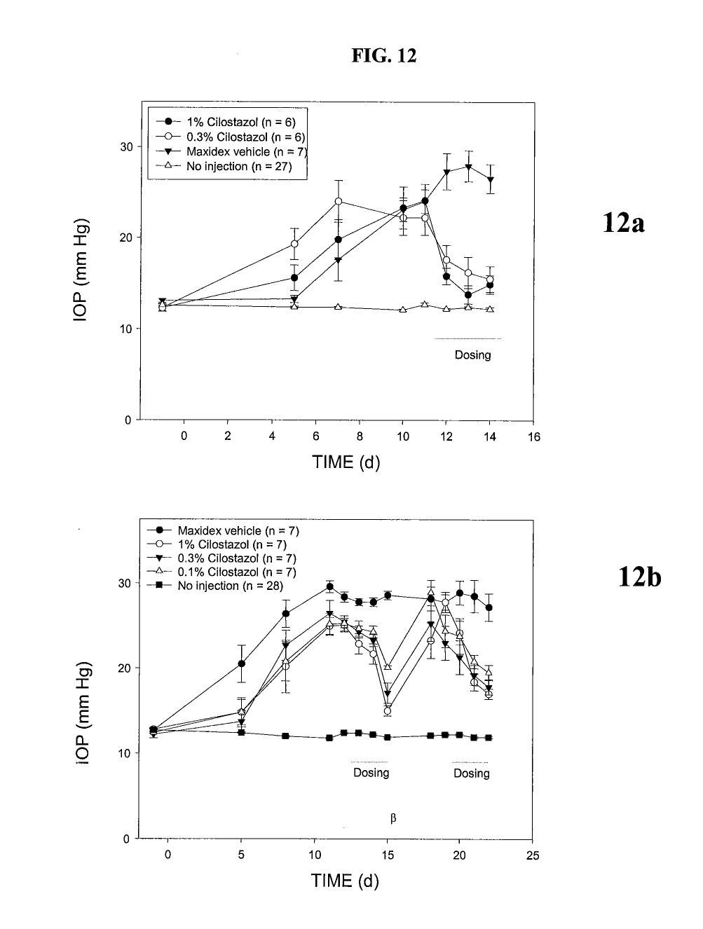

FIGS. 12a-12b are graphs showing the effect of topical ocular administration of various concentrations of cilostazol compared to control on Ad.TGFβ2-induced ocular hypertension in mice. Ad.TGFβ2 viral vector was injected on Day 0. Dosing with Cilostazol or vehicle was performed on the indicated days (denoted by horizontal bars). Intraocular pressure (IOP) was monitored in conscious mice using a rebound tonometer. The data presented in FIGS. 12a-12b depicts results from two independent studies and demonstrates substantial IOP-lowering effects from both the 0.3 and 1.0% cilostazol formulations relative to control (Maxidex vehicle).

### Methods for Examples 1-5

Human TM cell culture: Human TM cells were isolated from post-mortem human donor tissue, characterized, and cultured as previously described. Generation and characterization of the transformed (GTM-3) cell line was also as previously described (Pang et al., Preliminary characterization of a transformed cell strain derived from human trabecular meshwork., Curr. Eye Res., Vol. 13:51-63, 1994.)

PAI-1 ELISA: 24-well plates of TM cell cultures were serum-deprived for 24 h followed by an additional 24 h (or as indicated) incubation with TGFβ2 in a serum-free medium. Aliquots of supernatants from the treated cultures were quantified for secreted PAI-1 content by means of human PAI-1 ELISA kit (American Diagnostica).

TM cell adhesion: TM cell adhesion was determined by means of InnoCyte ECM Cell Adhesion Assay (Calbiochem). TM cells (20,000/well; serum-free medium) were plated onto a vitronectin-coated 96-well plate. Test agents were then added, followed by incubation in a cell culture incubator for the times indicated. Non-adherent cells were then removed by decantation and gentle wash of the wells with PBS. Relative cell attachment was determined by means of fluorescent dye (calcein-AM) uptake.

TM cell migration: Migration of TM cells was assessed using InnoCyte Cell Migration Assay (Calbiochem). TM cells (50,000/well; serum-free medium) were plated into the upper well assembly of the migration chamber supplied with the kits. Lower wells were filled with solutions of the test agents and the chamber was then incubated in a cell culture incubator. After 4 h, the upper well assembly was removed and supernatants were gently decanted to remove unattached cells. The upper well assembly was then placed into a fresh lower plate containing a mixture of detachment buffer and calcein-AM. 60 minutes later, aliquots from each lower well were transferred to a fresh 96 well plate and relative fluorescence determined.

### Example 6

| Ingredients | Concentration (w/v %) |
|---|---|
| Cilostazol | 0.01-2% |
| Hydroxypropyl methylcellulose | 0.5% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |
| Purified water | q.s. to 100% |

### Example 7

| Ingredients | Concentration (w/v %) |
|---|---|
| Cilostamide | 0.01-2% |
| Methyl cellulose | 4.0% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |
| Purified water | q.s. to 100% |

### Example 8

| Ingredients | Concentration (w/v %) |
|---|---|
| 3,4-dehydro Cilostazol | 0.01-2% |
| Guar gum | 0.4-6.0% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |
| Purified water | q.s. to 100% |

### Example 9

| Ingredients | Concentration (w/v %) |
|---|---|
| Cilostazol | 0.01-2% |
| White petrolatum and mineral oil and lanolin | Ointment consistency |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein.

What is claimed is:

1. A method for treating glaucoma or elevated IOP in a patient comprising:
administering to the patient an effective amount of a composition comprising cilostazol or an analog or metabolite thereof.

2. The method of claim 1 wherein said composition further comprises a compound selected from the group consisting of:
opthalmologically acceptable preservatives, surfactants, viscosity enhancers, penetration enhancers, gelling agents, hydrophobic bases, vehicles, buffers, sodium chloride, water, and combinations thereof.

3. The method of claim 1, further comprising administering, either as part of said composition or as a separate administration, a compound selected from the group consisting of:
β-blockers, prostaglandin analogs, carbonic anhydrase inhibitors, α₂ agonists, miotics, neuroprotectants, rho kinase inhibitors, and combinations thereof.

4. The method of claim 1 wherein said composition comprises from about 0.01 percent weight/volume to about 5 percent weight/volume of cilostazol or an analog or metabolite thereof.

5. The method of claim 1 wherein said composition comprises from about 0.25 percent weight/volume to about 2 percent weight/volume of cilostazol or an analog or metabolite thereof.

6. The method of claim 1 wherein said composition comprises cilostazol, 3,4-dehydro cilostazol or cilostamide.

7. The method of claim 1 wherein said composition further comprises an agent selected from the group consisting of:
ZK4044, PAI-039, WAY-140312, HP-129, T-686, XR5967, XR334, XR330, XR5118, PAI-1 antibodies, PAI-1 peptidomimetics, and combinations thereof.

8. A method of treating a PAI-1-associated ocular disorder in a subject in need thereof, comprising:
administering to the patient an effective amount of a composition comprising cilostazol or an analog or metabolite thereof.

9. The method of claim 8 wherein the subject has or is at risk of developing ocular hypertension or glaucoma.

10. The method of claim 8 wherein said administering reduces the amount of active PAI-1 in said subject.

11. The method of claim 8 wherein said composition further comprises a compound selected from the group consisting of:
opthalmologically acceptable preservatives, surfactants, viscosity enhancers, penetration enhancers, gelling agents, hydrophobic bases, vehicles, buffers, sodium chloride, water, and combinations thereof.

12. The method of claim 8, further comprising administering, either as part of said composition or as a separate administration, a compound selected from the group consisting of:
β-blockers, prostaglandin analogs, carbonic anhydrase inhibitors, α₂ agonists, miotics, neuroprotectants, rho kinase inhibitors, and combinations thereof.

13. The method of claim 8 wherein said composition comprises from about 0.01 percent weight/volume to about 5 percent weight/volume of cilostazol or an analog or metabolite thereof.

14. The method of claim 8 wherein said composition comprises from about 0.25 percent weight/volume to about 2 percent weight/volume of cilostazol or an analog or metabolite thereof.

15. The method of claim 8 wherein said composition comprises cilostazol, 3,4-dehydro cilostazol or cilostamide.

16. The method of claim 8 wherein said composition further comprises an agent selected from the group consisting of:
ZK4044, PAI-039, WAY-140312, HP-129, T-686, XR5967, XR334, XR330, XR5118, PAI-1 antibodies, PAI-1 peptidomimetics, and combinations thereof.
